(19) United States
(12) Patent Application Publication
(10) Pub. No.: US 2025/0210299 A1
(43) Pub. Date: Jun. 26, 2025

(54) PARTICLE BEAM TREATMENT APPARATUS, DEFLECTION MAGNET APPARATUS, AND PARTICLE BEAM ADJUSTMENT METHOD

(71) Applicant: Sumitomo Heavy Industries, Ltd., Tokyo (JP)

(72) Inventor: Yuta EBARA, Kanagawa (JP)

(73) Assignee: Sumitomo Heavy Industries, Ltd., Tokyo (JP)

(21) Appl. No.: 18/986,729

(22) Filed: Dec. 19, 2024

(30) Foreign Application Priority Data
Dec. 22, 2023 (JP) 2023-217071

(51) Int. Cl. H01J 37/147; A61N 5/10; H01J 37/141

(52) U.S. Cl. CPC H01J 37/1475; A61N 5/1048; A61N 5/1078; H01J 37/141; A61N 2005/1087

(57) ABSTRACT

A particle beam treatment apparatus includes an irradiation portion configured to irradiate a particle beam to an irradiation target, a transport portion configured to transport the particle beam, and a deflection magnet apparatus having a deflection magnet for deflecting the particle beam in the transport portion. The deflection magnet is rotatable around a beam axis of the particle beam. A deflection magnet apparatus includes a deflection magnet that deflects a particle beam. The deflection magnet is rotatable around a beam axis of the particle beam. A particle beam adjustment method for adjusting a deflection magnet that deflects a particle beam includes adjusting the particle beam by rotating the deflection magnet around a beam axis of the particle beam.

### CROSS-REFERENCE TO RELATED APPLICATION

This application claims the priority benefits of Japanese application no. 2023-217071, filed on Dec. 22, 2023. The entirety of the above-mentioned patent application is hereby incorporated by reference herein and made a part of this specification.

### BACKGROUND

### Technical Field

The disclosure relates to a particle beam treatment apparatus, a deflection magnet apparatus, and a particle beam adjustment method.

### Description of Related Art

Conventionally, as a particle beam treatment apparatus that performs treatment by irradiating a particle beam to a patient's affected area, for example, an apparatus described in Patent Document 1 (Japanese Patent Application Laid-Open (JP-A) No. 2017-209372) is known. In the particle beam treatment apparatus described in Patent Document 1, a particle beam is irradiated from an irradiation portion.

Here, the particle beam treatment apparatus includes a transport portion that transports the particle beam from an accelerator to the irradiation portion. A deflection magnet apparatus that deflects the particle beam is provided for such a transport portion. In this case, the deflection magnet apparatus may include a deflection magnet that adjusts in an x-axis direction and a deflection magnet that adjusts in a y-axis direction. By combining the deflection forces of such biaxial deflection magnets, the particle beam may be deflected and adjusted in a desired direction. However, in conventional particle beam treatment apparatuses, depending on the angle of deflection, the combined deflection force may become small. As a result, an effective deflection force may not be obtained depending on the angle.

Thus, the disclosure provides a particle beam treatment apparatus, a deflection magnet apparatus, and a particle beam adjustment method that may effectively obtain a deflection force regardless of the deflection direction of the particle beam.

### SUMMARY

The particle beam treatment apparatus according to one aspect of the disclosure includes an irradiation portion configured to irradiate a particle beam to an irradiation target; a transport portion configured to transport the particle beam; and a deflection magnet apparatus having a deflection magnet for deflecting the particle beam in the transport portion. The deflection magnet is rotatable around a beam axis of the particle beam.

The deflection magnet apparatus according to one aspect of the disclosure is a deflection magnet apparatus including a deflection magnet that deflects a particle beam. The deflection magnet may be rotatable around a beam axis of the particle beam.

A particle beam adjustment method according to one aspect of the disclosure is a particle beam adjustment method for adjusting a deflection magnet that deflects a particle beam in a transport portion. The particle beam is adjusted by rotating the deflection magnet around the beam axis of the particle beam.

### BRIEF DESCRIPTION OF THE DRAWINGS

FIG. 1 is a schematic configuration diagram showing the particle beam treatment apparatus according to an embodiment of the disclosure.

FIG. 2 is a schematic side view of the deflection magnet apparatus.

FIGS. 3A, 3B, and 3C are schematic diagrams showing the deflection magnet apparatus.

FIGS. 4A, 4B, and 4C are schematic diagrams showing the deflection magnet apparatus.

FIGS. 5A, 5B, and 5C are schematic diagrams showing the deflection magnet apparatus.

FIGS. 6A and 6B are schematic diagrams showing the deflection magnet apparatus.

FIGS. 7A and 7B are schematic diagrams showing an example of the adjusting mechanism.

FIGS. 8A and 8B are schematic diagrams showing an example of the adjusting mechanism.

FIGS. 9A and 9B are schematic diagrams showing an example of the adjusting mechanism.

### DESCRIPTION OF THE EMBODIMENTS

According to the particle beam treatment apparatus, the deflection magnet that deflects the particle beam of the transport portion is rotatable around the beam axis of the particle beam. In this case, the deflection magnet may be rotated around the beam axis to align with the desired deflection direction of the particle beam. Thus, the direction in which the deflection force of the deflection magnet is effectively obtained may be aligned with the desired deflection direction of the particle beam. As a result, effective deflection force may be obtained regardless of the deflection direction of the particle beam.

The deflection magnet apparatus has a pair of deflection magnets, and the pair of deflection magnets may be rotatable around a beam axis of the particle beam in a state where the deflection magnets are fixed to each other in a rotation direction. When combining the deflection forces of the pair of deflection magnets, there are angles at which the combined deflection force may be secured at a large magnitude and angles at which the deflection force becomes small. By rotating the pair of deflection magnets around the beam axis of the particle beam, it is possible to align the direction in which a large deflection force may be secured with the desired deflection direction of the particle beam. Thus, regardless of the deflection direction of the particle beam around the beam axis, a large deflection force of the pair of deflection magnets may be secured. Here, since the pair of deflection magnets are fixed to each other in the rotation direction, the adjustment of the rotation angle may be performed more easily compared to individually adjusting the rotation angles of the pair of deflection magnets.

The deflection magnet apparatus includes a pair of deflection magnets, and one of the pair of deflection magnets may be rotatable around the beam axis of the particle beam independently from the other. When combining the deflection forces of the pair of deflection magnets, there are angles at which the combined deflection force may be secured at a large magnitude and angles at which the deflection force becomes small. By rotating the pair of deflection magnets around the beam axis of the particle beam, it is possible to align the direction in which a large deflection force may be secured with the desired deflection direction of the particle beam. Thus, regardless of the deflection direction of the particle beam around the beam axis, a large deflection force of the pair of deflection magnets may be secured. Here, when combining the deflection force of one deflection magnet with the deflection force of the other deflection magnet, there exists a directional component where the two deflection forces cancel each other out. In response to this, one of the pair of deflection magnets is rotatable around the beam axis of the particle beam independently from the other. As a result, the relative rotation angle of the deflection magnets may be adjusted to minimize the directional components that cancel each other out. This allows for effective utilization of energy applied to the deflection magnet apparatus.

The deflection magnet apparatus includes one deflection magnet, and the one deflection magnet may be rotatable around the beam axis of the particle beam. In this case, the number of magnets in the deflection magnet apparatus may be reduced.

The particle beam treatment apparatus may include an adjusting mechanism for adjusting the rotation angle of the deflection magnet with respect to the beam axis. In this case, by using the adjusting mechanism, the rotation angle of the deflection magnet may be adjusted easily and accurately.

The adjusting mechanism may include a driver for rotating the deflection magnet with respect to the beam axis. In this case, the rotation angle of the deflection magnet may be adjusted without manual operation by an operator.

The beam axis extends in a horizontal direction, and an orientation of magnetic field of the deflection magnet may be inclined with respect to a vertical direction.

The particle beam treatment apparatus may further include a controller configured to control the deflection magnet apparatus. The controller may perform a first adjustment of the particle beam by rotating the deflection magnet. In this case, the controller may roughly adjust the direction of the deflection force by rotating the deflection magnet itself.

The controller may perform a second adjustment of the particle beam by electrically controlling the deflection magnet. In this case, the controller may fine-tune the direction of the deflection force.

The deflection magnet apparatus may include a pair of deflection magnets, and in the first adjustment, the controller may rotate the deflection magnet relative to each other. In this case, the controller may also adjust the angle between the pair of deflection magnets in the first adjustment.

The deflection magnet apparatus may deflect the particle beam in a desired direction by being placed where the particle beam passes. At this time, the deflection magnet may be rotated around the beam axis to align with the desired deflection direction of the particle beam. Thus, the direction in which the deflection force of the deflection magnet is effectively obtained may be aligned with the desired deflection direction of the particle beam. As a result, effective deflection force may be obtained regardless of the deflection direction of the particle beam.

According to the particle beam adjustment method, the particle beam may be deflected in a desired direction by providing the deflection magnet where the particle beam passes. At this time, the deflection magnet is rotated around the beam axis to align with the desired deflection direction of the particle beam. Thus, the direction in which the deflection force of the deflection magnet is effectively obtained may be aligned with the desired deflection direction of the particle beam. As a result, effective deflection force may be obtained regardless of the deflection direction of the particle beam.

The disclosure may provide a particle beam treatment apparatus, a deflection magnet apparatus, and a particle beam adjustment method that may effectively obtain deflection force regardless of the deflection direction of the particle beam.

The following describes a particle beam treatment apparatus related to an embodiment of the disclosure with reference to the attached drawings. In the description of the drawings, the same elements are given the same reference numerals, and duplicate descriptions are omitted.

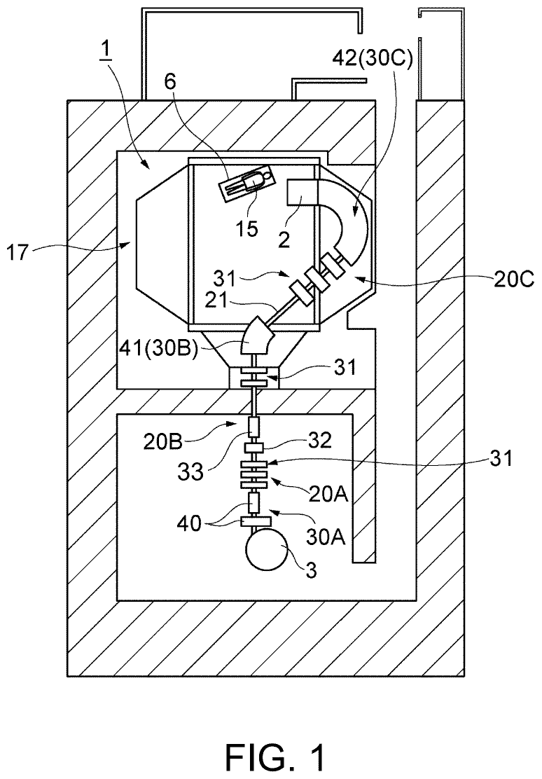

FIG. 1 is a schematic configuration diagram showing the particle beam treatment apparatus 1 according to an embodiment of the disclosure. The particle beam treatment apparatus 1 is a system used for cancer treatment and the like by radiation therapy. The particle beam treatment apparatus 1 may be an irradiation apparatus related to a scanning method. It is noted that the scanning method is not particularly limited, and line scanning, raster scanning, spot scanning, etc., may be adopted. The particle beam treatment apparatus 1 includes an accelerator 3 that accelerates charged particles generated in an ion source apparatus and emits them as a particle beam, an irradiation portion 2 that irradiates the particle beam to a patient 15, and a transport portion 20 that transports the particle beam emitted from the accelerator 3 to the irradiation portion 2. The irradiation portion 2 is attached to a rotary gantry 17 that is provided so as to surround a treatment table 6. The irradiation portion 2 is made rotatable around the treatment table 6 on which the patient 15 is positioned, with the central axis as the rotation center by the rotary gantry 17.

The accelerator 3 is an apparatus that accelerates charged particles and emits a particle beam B with a predetermined energy. Examples of the accelerator 3 include a cyclotron, a synchrocyclotron, and the like. The particle beam B generated in the accelerator 3 is transported to the irradiation portion 2 by the transport portion 20. The transport portion 20 connects the accelerator 3 and the irradiation portion 2, and transports the particle beam B emitted from the accelerator 3 to the irradiation portion 2. The detailed configuration of the transport portion 20 is described later.

The irradiation portion 2 is designed to irradiate a particle beam to a tumor inside the patient 15's body. A particle beam refers to charged particles accelerated to high speeds, such as proton beams, heavy particle (heavy ion) beams, electron beams. Specifically, the irradiation portion 2 is an apparatus that irradiates the particle beam, which has been emitted from the accelerator 3 that accelerates charged particles generated in an ion source (not shown) and transported by the transport portion 20, to the tumor. The irradiation portion 2 has a scanning electromagnet. The irradiation portion 2 may also include quadrupole electromagnets, monitors, degraders, etc. The scanning electromagnet changes the magnetic field between a pair of electromagnets according to the current supplied from the controller, and scans the particle beam passing between these electromagnets. It is noted that the scanning electromagnet scans the particle beam B so that the particle beam is irradiated according to a scan pattern planned in advance by a treatment planning apparatus.

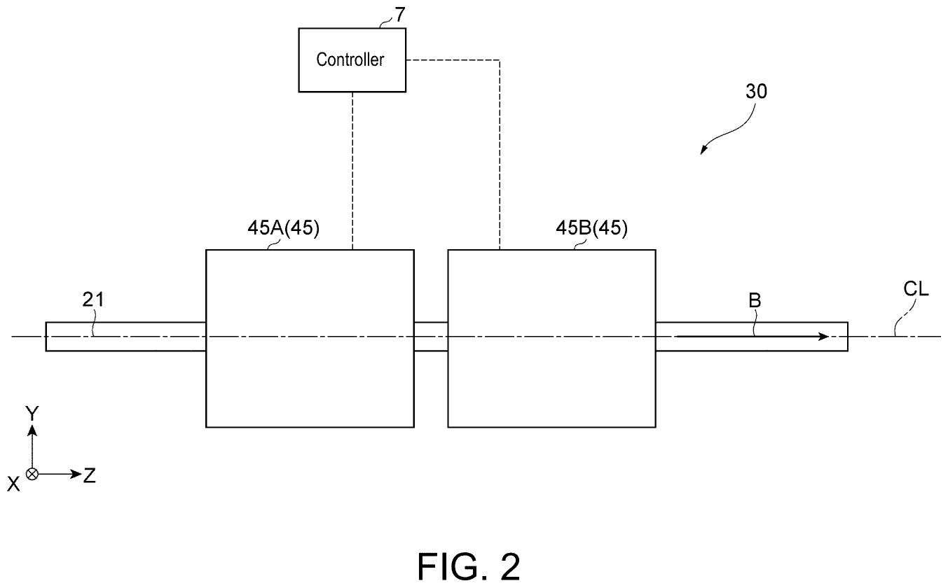

The controller 7 (refer to FIG. 2) is composed of, for example, a CPU, ROM, and RAM. This controller 7 controls the accelerator 3, the transport portion 20, and the irradiation portion 2, etc., based on the detection results output from each monitor.

Further, the controller 7 of the particle beam treatment apparatus 1 is connected to a treatment planning apparatus that performs treatment planning for particle beam treatment. The treatment planning apparatus measures the patient 15's tumor using CT or the like before treatment, and plans the dose distribution (the dose distribution of the particle beam to be irradiated) at each position of the tumor. Specifically, the treatment planning apparatus creates a scan pattern for the tumor. The treatment planning apparatus transmits the created scan pattern to the controller 7. In the scan pattern created by the treatment planning apparatus, it is planned what scanning path the particle beam traces and at what scanning speed.

In the case of irradiating a particle beam using the scanning method, the tumor is virtually divided into multiple layers, and the particle beam is scanned and irradiated in one layer according to the scanning path determined in the treatment planning. Then, after the irradiation of the particle beam in that one layer is completed, the irradiation of the particle beam in the adjacent next layer is performed.

The transport portion 20 has a beam duct 21 (transport portion) that transports the particle beam B, and multiple electromagnets. The transport portion 20 includes a BTS (beam transport system) system 20A, an ESS (energy selection system) system 20B, and a GTS (gantry transport system) system 20C. The BTS system 20A is a system that transports the particle beam B. The ESS system 20B is a system that selects the energy of the particle beam B. The GTS system 20C is a transport system for the particle beam B in the rotary gantry 17. The transport portion 20 includes a deflection magnet apparatus 30 in each system.

The components in each system are described. However, the components shown in FIG. 1 are only an example and may be modified as appropriate. The BTS system 20A mainly includes a deflection magnet apparatus 30A and focusing electromagnets 31 and 31. The deflection magnet apparatus 30A has a deflection magnet 40. The deflection magnet 40 is an electromagnet that deflects the particle beam transported in the beam duct 21. In this example, the deflection magnet apparatus 30A includes a pair of deflection magnets 40, enabling biaxial adjustment of the particle beam. However, as described later, the deflection magnet apparatus 30A may have one deflection magnet 40. The focusing electromagnet 31 is an electromagnet that focuses the particle beam.

The ESS system 20B includes a degrader 32 and a collimator apparatus 33. The degrader 32 adjusts the range of the particle beam B by reducing the energy of the passing particle beam B. The collimator apparatus 33 is a member that collimates the particle beam.

The GTS system 20C includes a focusing electromagnet 31, a deflection magnet apparatus 30B, a focusing electromagnet 31, and a deflection magnet apparatus 30C. The deflection magnet apparatus 30B includes a deflection magnet 41 that bends the particle beam B, which has passed through the ESS system 20B, towards the outer periphery side of the rotary gantry 17. The deflection magnet apparatus 30C includes a deflection magnet 42 that is bent from the outer periphery side of the rotary gantry 17 to the irradiation portion 2 on the inner periphery side.

Here, the deflection magnets 41 and 42 are electromagnets for bending the particle beam B to such an extent that it becomes necessary to change the angle of the beam duct 21. The deflection magnets 41 and 42 may bend the particle beam B to a desired angle. On the other hand, the deflection magnet 40 performs adjustment by deflecting the particle beam B within a range that may maintain the angle of the beam duct 21. Although not particularly limited, the angular range of deflection by the deflection magnet 40 may be 45° or less, or 90° or less.

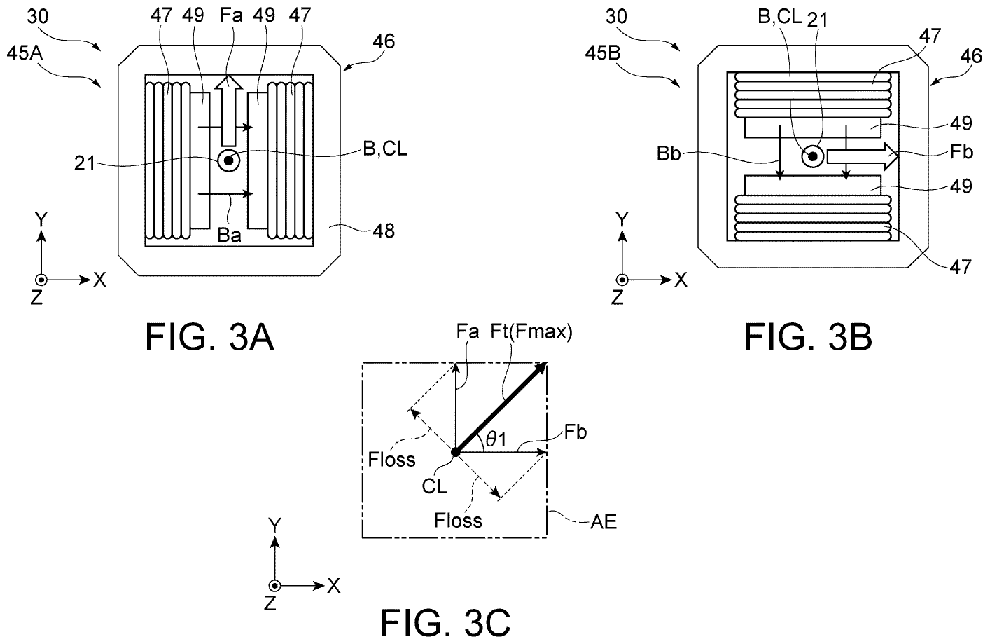

Next, referring to FIG. 2 and FIGS. 3A, 3B, and 3C, the detailed configuration of the deflection magnet apparatus 30 is described. It is noted that in the following description, the direction in which the particle beam B travels is defined as the z-axis direction. The direction perpendicular to the z-axis direction is defined as the x-axis direction, and the direction perpendicular to both the z-axis direction and the x-axis direction is defined as the y-axis direction. The central axis of the beam duct is defined as the beam axis CL of the particle beam B. FIG. 2 is a schematic diagram of the deflection magnet apparatus 30 viewed from the x-axis direction. The deflection magnet apparatus 30 shown in FIG. 2 includes a deflection magnet 45A and a deflection magnet 45B. FIG. 3A is a view of the deflection magnet 45A viewed from the z-axis direction. FIG. 3B is a view of the deflection magnet 45B viewed from the z-axis direction. FIG. 3C is a diagram for describing the deflection force by the deflection magnet 45A and the deflection force by the deflection magnet 45B. It is noted that the deflection magnets 45A and 45B shown in FIGS. 3A, 3B, and 3C are defined as the “reference attitude”. It is noted that the orientation of the deflection force F due to the deflection action of the deflection magnets 45A and 45B is defined by the orientation of the Lorentz force determined by the orientation of the magnetic field at the beam axis CL and the orientation of the particle beam B. Here, it is assumed that the particle beam B is traveling along the beam axis CL.

As shown in FIGS. 3A and 3B, the deflection magnets 45A and 45B include a magnetic pole 46 and coils 47. The magnetic pole 46 includes a rectangular ring-shaped main body portion 48 surrounding the beam axis CL and a pair of protrusions 49. The main body portion 48 is arranged such that its central axis coincides with the beam axis CL. As shown in FIG. 3A, in the reference attitude of the deflection magnet 45A, the pair of protrusions 49 extend from the wall portion of the main body portion 48 facing the x-axis direction towards the beam axis CL. The pair of protrusions 49 are arranged to face each other in the x-axis direction, sandwiching the beam duct 21 from two sides in the x-axis direction. The coil 47 is provided on each protrusion 49. As a result, in the reference attitude, the deflection magnet 45A generates a magnetic field Ba in the x-axis direction. As a result, the deflection magnet 45A generates a deflection force Fa in the y-axis direction.

As shown in FIG. 3B, in the reference attitude of the deflection magnet 45B, the pair of protrusions 49 extend from the wall portion of the main body portion 48 facing the y-axis direction towards the beam axis CL. The pair of protrusions 49 are arranged to face each other in the y-axis direction, sandwiching the beam duct 21 from two sides in the y-axis direction. The coil 47 is provided on each protrusion 49. As a result, in the reference attitude, the deflection magnet 45B generates a magnetic field Bb in the x-axis direction. As a result, the deflection magnet 45B generates a deflection force Fb in the x-axis direction.

As shown in FIG. 3C, in the reference attitude, the deflection force Fa and the deflection force Fb form a 90° angle with each other. In the case where the magnitudes of the deflection forces Fa and Fb are equal, the combined deflection force Ft, which is the superposition of both forces, acts in a direction that forms a 450 angle with respect to the x-axis direction. It is noted that in FIGS. 3A, 3B, and 3C, the orientation of the deflection force Fb is on the positive side of the x-axis direction, but it may be changed to the negative side of the x-axis direction by changing the orientation of the current flowing through the coil 47. The orientation of the deflection force Fa is on the positive side of the y-axis direction, but it may be changed to the negative side of the y-axis direction by changing the orientation of the current flowing through the coil 47. Further, the magnitudes of the deflection forces Fa and Fb may be changed by adjusting the magnitude of the current flowing through the coil 47. The deflection magnet apparatus 30 may adjust the magnitude and direction of the combined deflection force Ft by adjusting the magnitudes and orientations of the deflection forces Fa and Fb. The direction of the combined deflection force Ft may be adjusted 360° around the beam axis CL. However, in the case of changing the orientation of the combined deflection force Ft while maintaining the reference attitude, when the magnitudes of the deflection forces Fa and Fb are maximized, the angle θ1 becomes maximum at 45°, 135°, 225°, and 315°. The combined deflection force Ft at this time may be referred to as the “maximum deflection force Fmax”. It is noted that the angle θ1 of the combined deflection force Ft is the angle based on the positive side of the x-axis direction. To set the combined deflection force Ft to angles other than those mentioned above, it is necessary to reduce either the deflection force Fa or Fb. Thus, the magnitude of the combined deflection force Ft for angles θ1 other than the above-mentioned angles becomes smaller than the magnitude of the maximum deflection force. In the case where the angle θ1 is 0°, the deflection force Fa becomes 0, so the combined deflection force Ft becomes only the magnitude of the deflection force Fb. In the case where the angle θ1 is 90°, the deflection force Fb becomes 0, so the combined deflection force Ft becomes only the magnitude of the deflection force Fa. Thus, in the case of a structure (referred to as a comparative example) where the angle of the deflection magnets 45A and 45B around the beam axis CL cannot be changed from the reference attitude, the maximum value of the combined deflection force Ft becomes smaller than the maximum deflection force Fmax for angles other than “angle θ1=45°, 135°, 225°, 315°”. The adjustment range AE of the combined deflection force Ft is shown in FIG. 3C. It is noted that the adjustment range AE related to the comparative example is fixed around the beam axis CL.

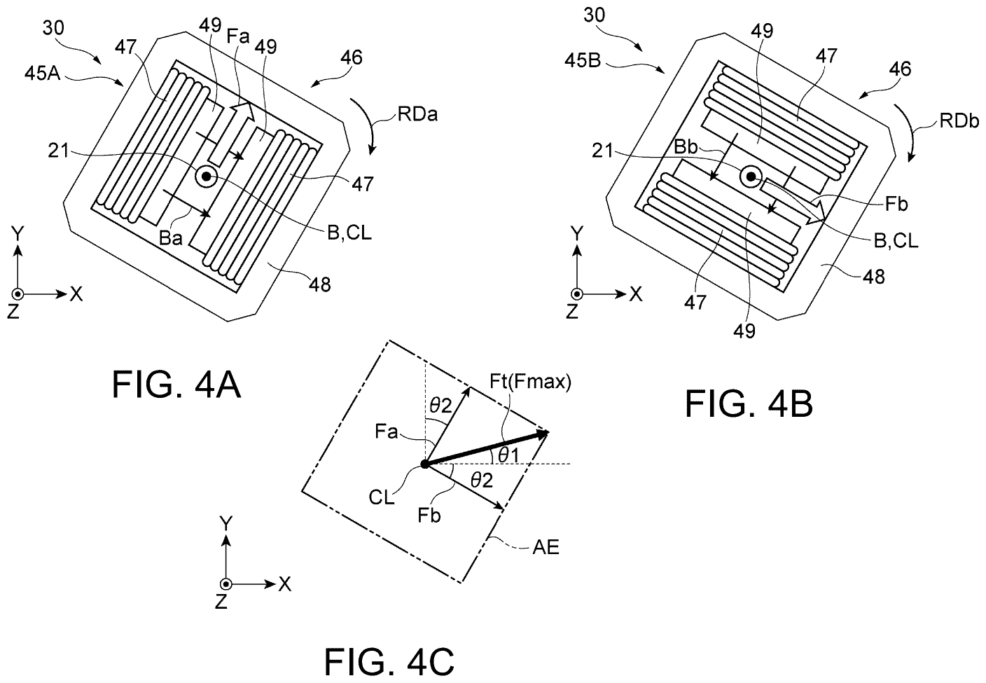

In contrast, the deflection magnet apparatus 30 according to this embodiment, as shown in FIGS. 4A, 4B, and 4C, includes deflection magnets 45A and 45B that are rotatable around the beam axis CL of the particle beam B. The deflection magnets 45A and 45B rotate with the beam axis CL as the rotation center. As a result, the deflection magnets 45A and 45B may change the directions of the deflection forces Fa and Fb with respect to the reference attitude.

As a configuration for rotating the deflection magnets 45A and 45B, the structure shown in FIGS. 4A, 4B, and 4C may be adopted. As shown in FIGS. 4A and 4B, the pair of deflection magnets 45A and 45B may be rotatable around the beam axis CL of the particle beam B while being fixed to each other in the rotation direction. In other words, the pair of deflection magnets 45A and 45B rotate around the beam axis CL in an integrated state. In this case, the orientation and rotation angle of the rotation direction RDa of the deflection magnet 45A and the orientation and rotation angle of the rotation direction RDb of the deflection magnet 45B become identical. The deflection force Fa by the deflection magnet 45A acts in a direction rotated by the rotation angle with respect to the y-axis direction. The deflection force Fb by the deflection magnet 45B acts in a direction rotated by the rotation angle with respect to the x-axis direction. However, the relationship where the deflection force Fa and the deflection force Fb form 90° with each other is maintained regardless of the rotation angle and orientation.

The method for fixing the pair of deflection magnets 45A and 45B to each other in the rotation direction is not particularly limited. For example, the body portion 48 of the deflection magnet 45A and the body portion 48 of the deflection magnet 45B may be fixed to each other by a predetermined connecting member. For instance, the deflection magnets 45A and 45B may be fixed to a common base member. Alternatively, a pin may be inserted into the body portions 48 of the deflection magnets 45A and 45B to fix them.

In this case, as shown in FIG. 4C, the direction of the deflection force Fa may be changed by the rotation angle θ2 from the y-axis direction, and the direction of the deflection force Fb may be changed by the rotation angle θ2 from the x-axis direction. In this case, the maximum deflection force Fmax may be generated at angles other than “angle θ1=45°”. It is noted that in the case where the adjustment range of the rotation angle θ2 is set to at least 90°, by adjusting the orientations of the deflection forces Fa and Fb, the maximum deflection force Fmax may be generated in the range of “angle θ1=0° to 360°”. In other words, the deflection magnet apparatus 30 may adjust the magnitude of the combined deflection force Ft in the range of “0 to maximum deflection force Fmax” all around the beam axis CL. Thus, the adjustment range AE is not fixed around the beam axis CL, but is set to a range rotated around the beam axis CL. However, in the case where the adjustment range of the particle beam B is somewhat limited, the adjustment range of the rotation angle θ2 only needs to be greater than 0° and may be less than 90°.

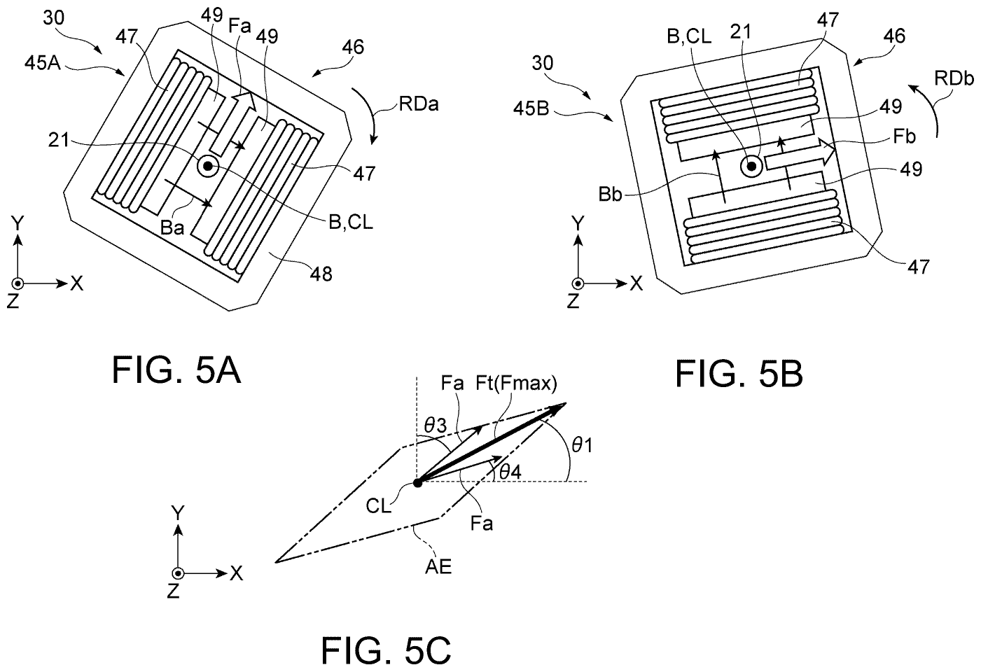

As a configuration for rotating the deflection magnets 45A and 45B, the structure shown in FIGS. 5A, 5B, and 5C may be adopted. As shown in FIGS. 5A and 5B, one of the pair of deflection magnets 45A and 45B may rotate around the beam axis CL of the particle beam B independently from the other. In the example shown in FIGS. 5A and 5B, the pair of deflection magnets 45A and 45B may rotate around the beam axis CL of the particle beam B independently of each other. In other words, the pair of deflection magnets 45A and 45B rotate around the beam axis CL in a state separated from each other. In this case, the orientation and rotation angle of the rotation direction RDa of the deflection magnet 45A and the orientation and rotation angle of the rotation direction RDb of the deflection magnet 45B do not have to be identical. The deflection force Fa by the deflection magnet 45A acts in a direction rotated by the rotation angle with respect to the y-axis direction. The deflection force Fb by the deflection magnet 45B acts in a direction rotated by the rotation angle with respect to the x-axis direction. Unlike the structure in FIGS. 4A, 4B, and 4C, the angle between the deflection force Fa and the deflection force Fb may be other than 90°.

In this case, as shown in FIG. 5C, the direction of the deflection force Fa may be changed by the rotation angle θ3 from the y-axis direction, and the direction of the deflection force Fb may be changed by the rotation angle θ4 from the x-axis direction. The rotation angle θ3 and the rotation angle θ4 may be different from each other. In this case, the maximum deflection force Fmax may be generated at angles other than “angle θ1=45°”. It is noted that in the case where the adjustment range of the rotation angles θ3 and θ4 is set to at least 90°, by adjusting the orientations of the deflection forces Fa and Fb, the maximum deflection force Fmax may be generated in the range of “angle θ1=0° to 360°”. In other words, the deflection magnet apparatus 30 may adjust the magnitude of the combined deflection force Ft in the range of “0 to maximum deflection force Fmax” all around the beam axis CL. The adjustment range AE is not limited to a square shape as shown in FIG. 4C, but may be any quadrilateral shape according to the directions of the deflection forces Fa and Fb, as shown in FIG. 5C. However, in the case where the adjustment range of the particle beam B is somewhat limited, the adjustment range of the rotation angles θ3 and θ4 only needs to be greater than 0° and may be less than 90°.

In this case, as shown in FIG. 3C, the angle formed between the deflection force Fa and the maximum deflection force Fmax, and the angle formed between the deflection force Fb and the maximum deflection force Fmax are fixed at 45°. As a result, among the deflection force Fa and deflection force Fb, the component Floss that does not contribute to the maximum deflection force Fmax and cancels each other out becomes larger. This component Floss also occurs in the structure shown in FIGS. 4A, 4B, and 4C. On the other hand, in the structure shown in FIGS. 5A, 5B, and 5C, unlike the structure in FIGS. 4A, 4B, and 4C, the angle between the deflection force Fa and the deflection force Fb may be smaller than 90°. In this case, as shown in FIG. 5C, the angle formed between the deflection force Fa and the maximum deflection force Fmax, and the angle formed between the deflection force Fb and the maximum deflection force Fmax may be made smaller than 45°. In this case, it is possible to reduce the component Floss that cancels each other out among the deflection force Fa and deflection force Fb, and increase the directional component that contributes to the maximum deflection force Fmax. Thus, the deflection magnet apparatus 30 shown in FIGS. 5A, 5B, and 5C may increase the maximum deflection force Fmax compared to the comparative example and the structure in FIGS. 4A, 4B, and 4C.

It is noted that in the case where the direction for adjusting the particle beam B may be predicted in advance, it is possible to operate by fixing one of the deflection magnets 45A and 45B and rotating the other. For example, in the case where the deflection magnet apparatus 30 is configured near the exit of the accelerator 3, the angle in the vertical direction of the particle beam B does not deviate significantly, but it may deviate significantly in the horizontal direction. In this case, the deflection magnet 45B in the horizontal direction may be fixed, and the other deflection magnet 45A may be made rotatable.

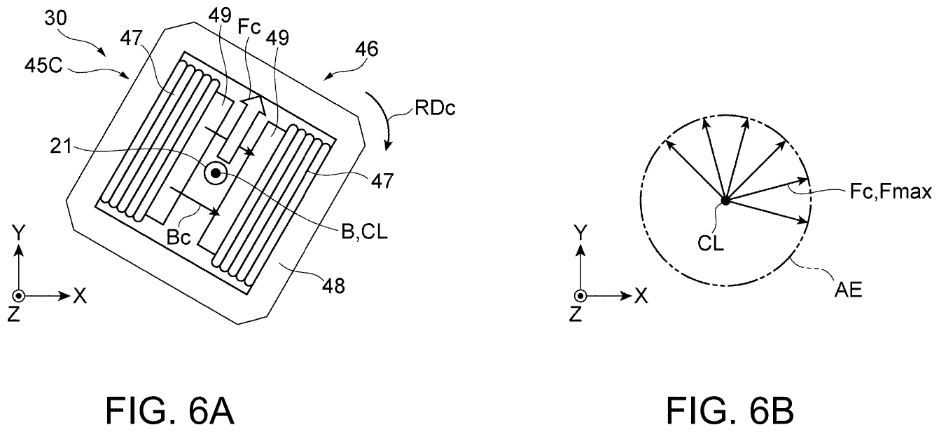

As shown in FIG. 6A, the deflection magnet apparatus 30 may include one deflection magnet 45C, and the one deflection magnet 45C may be rotatable around the beam axis CL of the particle beam B. Even with one deflection magnet 45C, it is possible to secure two degrees of freedom (angle and magnitude) as the degree of freedom of the deflection force Fc for adjusting the particle beam B. As shown in FIG. 6B, the deflection magnet apparatus 30 sets the maximum value of the deflection force Fc of the one deflection magnet 45C as the maximum deflection force Fmax. The direction of this maximum deflection force Fmax may be deflected around the beam axis CL. Thus, the adjustment range AE becomes circular.

In the deflection magnet apparatus 30 shown in FIG. 3A to FIG. 6B, the direction in which the beam axis CL extends is not particularly limited, and it may extend in the horizontal direction, in a direction inclined from the horizontal direction, or in the vertical direction. In the case where the beam axis CL extends in the horizontal direction, the z-axis direction and x-axis direction become horizontal directions, and the y-axis direction becomes the vertical direction. In the comparative example, the orientation of the magnetic field of the deflection magnet 45A (the orientation of the magnetic field on the beam axis CL), that is, the opposing direction of the protrusions 49, becomes the horizontal direction. Further, the orientation of the magnetic field of the deflection magnet 45B, that is, the opposing direction of the protrusions 49, becomes the vertical direction. In contrast, as shown in FIG. 4A to FIG. 6B, in the case where the deflection magnet 45B is adjusted by rotating around the beam axis CL, the orientation of the magnetic field of the deflection magnet 45B is arranged to be inclined with respect to the vertical direction. The orientation of the magnetic field of the deflection magnet 45A is arranged to be inclined with respect to the vertical direction, rather than the direction perpendicular to the vertical direction (i.e., horizontal direction).

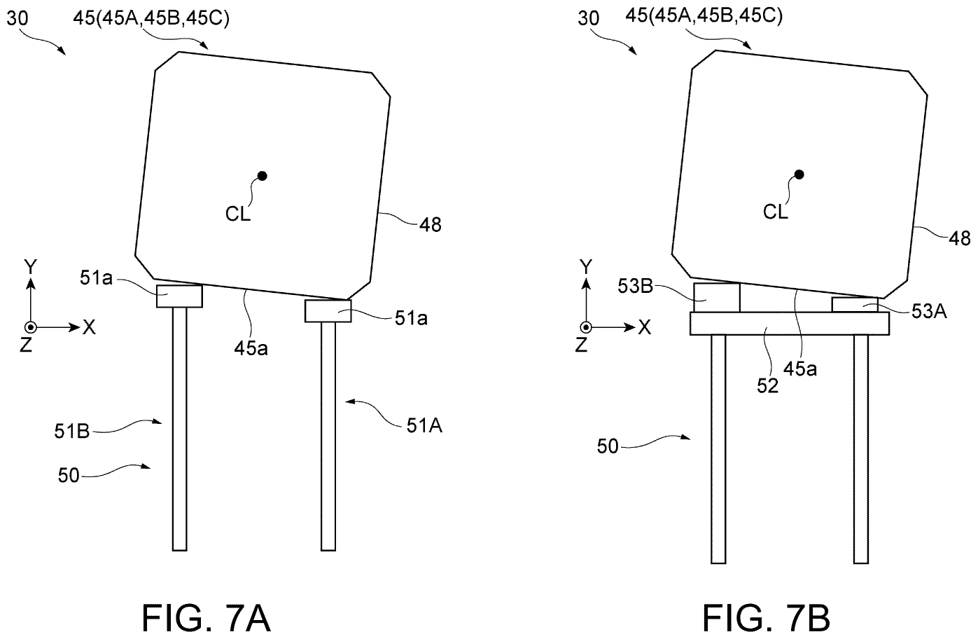

Next, referring to FIG. 7A to FIG. 9B, a mechanism for rotating the deflection magnet 45 is described. It is noted that the following mechanism may be applied to any of the deflection magnets 45A, 45B, and 45C. As shown in FIG. 7A to FIG. 9B, the deflection magnet apparatus 30 includes an adjusting mechanism 50 that adjusts the rotation angle of the deflection magnet 45 with respect to the beam axis CL.

FIGS. 7A and 7B show examples where the y-axis direction is the vertical direction. In other words, gravity is assumed to act towards the negative side of the y-axis direction. The adjusting mechanism 50 shown in FIG. 7A includes support portions 51A and 51B that support the deflection magnet 45 from below. The support portions 51A and 51B are arranged to be separated from each other in the x-axis direction, and respectively support two end sides of the bottom surface 45a of the deflection magnet 45 in the x-axis direction. The support portions 51A and 51B have mounting portions 51a on which the bottom surface 45a of the deflection magnet 45 is placed. The adjusting mechanism 50 adjusts the rotation angle of the deflection magnet 45 by adjusting the height of the mounting portion 51a of the support portion 51A and the height of the mounting portion 51a of the support portion 51B. The adjusting mechanism 50 may set the deflection magnet 45 to the reference attitude (refer to FIGS. 3A, 3B, and 3C) by making the heights of the mounting portions 51a of the support portions 51A and 51B the same. On the other hand, the adjusting mechanism 50 rotates the deflection magnet 45 and maintains its attitude at the rotated position by providing a difference in the heights of the mounting portions 51a of the support portions 51A and 51B.

The adjusting mechanism 50 shown in FIG. 7B includes a base member 52 that extends parallel to the XZ plane below the deflection magnet 45, and spacers 53A and 53B configured on the base member 52. The spacers 53A and 53B are arranged to be separated from each other in the x-axis direction, and respectively support two end sides of the bottom surface 45a of the deflection magnet 45 in the x-axis direction. The adjusting mechanism 50 adjusts the rotation angle of the deflection magnet 45 by adjusting the thickness of the spacer 53A and the thickness of the spacer 53B. The adjusting mechanism 50 may set the deflection magnet 45 to the reference attitude (refer to FIGS. 3A, 3B, and 3C) by making the thicknesses of the spacers 53A and 53B the same. On the other hand, the adjusting mechanism 50 rotates the deflection magnet 45 and maintains its attitude at the rotated position by providing a difference in the thicknesses of the spacers 53A and 53B.

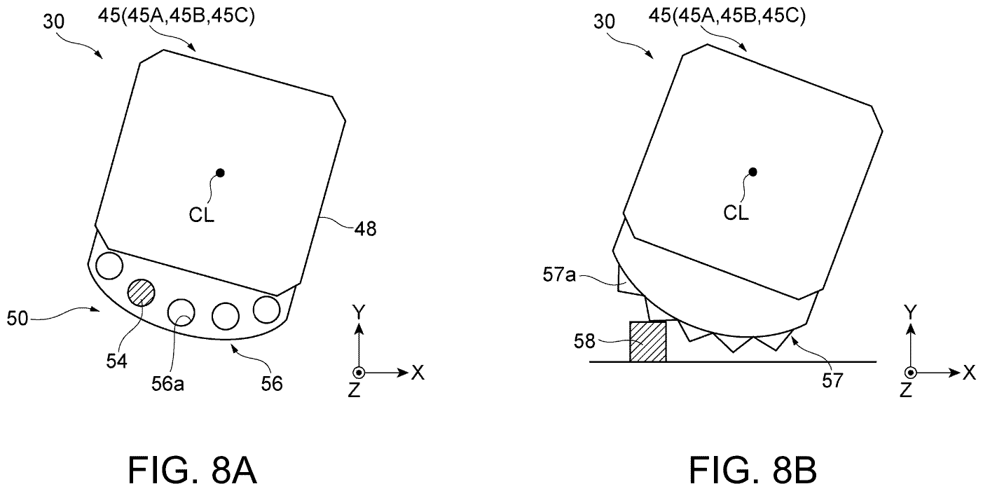

As shown in FIG. 8A, an adjusting mechanism 50 using a pin 54 may be adopted. As shown in FIG. 8A, the adjusting mechanism 50 includes a receiving portion 56 provided on the negative side of the deflection magnet 45 in the y-axis direction, and a pin 54. The receiving portion 56 is formed with multiple receiving holes 56a arranged at a predetermined pitch around the beam axis CL. The receiving holes 56a receive the pin 54. It is noted that the receiving holes 56a and the pin 54 extend in the z-axis direction. In the adjusting mechanism 50, the deflection magnet 45 and the receiving portion 56 are rotated to a desired angle. In this case, the pin 54 is inserted into any of the receiving holes 56a of the receiving portion 56. At this time, on the inner side of the receiving hole 56a, it communicates with another receiving hole formed in another base member or the like. Thus, by the pin 54 being inserted through the receiving hole 56a and the other receiving hole, the pin 54 fixes the deflection magnet 45 at the rotated position together with the receiving portion 56.

As shown in FIG. 8B, an adjusting mechanism 50 using an engagement portion 57a may be adopted. As shown in FIG. 8B, the adjusting mechanism 50 includes an engagement member 57 provided on the negative side of the deflection magnet 45 in the y-axis direction, and a support member 58. The engagement member 57 has multiple engagement portions 57a arranged at a predetermined pitch around the beam axis CL at the end portion on the negative side in the y-axis direction. The multiple engagement portions 57a are composed of a repeating pattern of peaks and valleys. In the adjusting mechanism 50, the deflection magnet 45 and the engagement member 57 are rotated to a desired angle. In this case, the support member 58 is engaged with any of the engagement portions 57a of the engagement member 57. At this time, the deflection magnet 45 is fixed at the rotated position together with the engagement member 57 by being supported in a state where the engagement portion 57a is engaged with the support member 58.

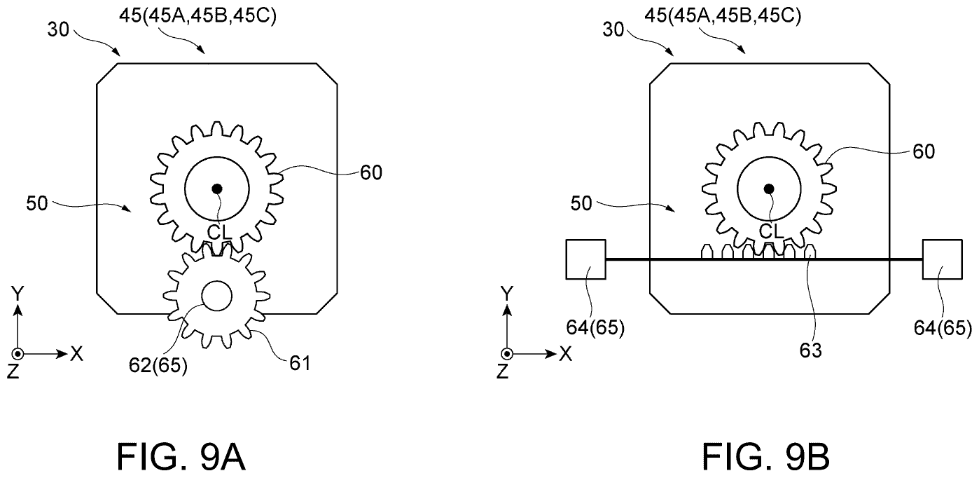

As shown in FIG. 9A, an adjusting mechanism 50 using a gear 60 may be adopted. As shown in FIG. 9A, the adjusting mechanism 50 includes a gear 60 fixed to the deflection magnet 45, and a gear 61 meshing with the gear 60. The gear 61 is fixed to a rotation shaft 62 and rotates together with the rotation shaft 62. The adjusting mechanism 50 rotates the deflection magnet 45 via the gear 60 by rotating the gear 61. The gear 61 is stopped at a position where the deflection magnet 45 reaches a desired rotation angle. As a result, the deflection magnet 45 maintains its attitude at the position of that rotation angle. By using the gear 60 in this manner, the rotation angle may be adjusted steplessly.

As shown in FIG. 9B, an adjusting mechanism 50 using a pinion gear 63 may be adopted. As shown in FIG. 9B, the adjusting mechanism 50 includes a gear 60 fixed to the deflection magnet 45, and a pinion gear 63 meshing with the gear 60. The pinion gear 63 is fixed to a rotation shaft 62 and rotates together with the rotation shaft 62. The adjusting mechanism 50 rotates the deflection magnet 45 via the gear 60 by moving the pinion gear 63 in the x-axis direction. The pinion gear 63 is stopped at a position where the deflection magnet 45 reaches a desired rotation angle. As a result, the deflection magnet 45 maintains its attitude at the position of that rotation angle. By using the gear 60 in this manner, the rotation angle may be adjusted steplessly.

The adjusting mechanism 50 may include a driver 65 for rotating the deflection magnet 45 with respect to the beam axis CL. In the example shown in FIG. 9A, the adjusting mechanism 50 may have a driver 65 such as a motor for rotating the rotation shaft 62. In the example shown in FIG. 9B, the adjusting mechanism 50 may have a driving mechanism 64 as the driver 65 for reciprocating the pinion gear 63 in the x-axis direction. It is noted that the gear 61 in FIG. 9A and the pinion gear 63 in FIG. 9B may be moved manually by an operator without using the driver 65. It is noted that the support portions 51A and 51B in FIG. 7A may be adjusted manually by an operator or adjusted by a driver. The rotation of the deflection magnet 45 and the operation of fixing it at that rotation angle as shown in FIG. 7B and FIGS. 8A and 8B may be performed manually by an operator, or may be operated by a driver with a mechanism provided for operation.

The adjustment of the deflection of the particle beam B by the deflection magnet apparatus 30 may be performed by the controller 7 (refer to FIG. 2). It is noted that the controller 7 may control the rotation angle of the deflection magnet 45 by controlling the aforementioned driver. The controller 7 may perform a two-stage adjustment for the structure shown in FIGS. 4A, 4B, and 4C and FIGS. 5A, 5B, and 5C. The controller 7 performs the first adjustment of the particle beam B by rotating the deflection magnet 45. In the first adjustment, a coarse adjustment is performed to adjust the approximate angle. The controller 7 performs the second adjustment of the particle beam B by electrically controlling the deflection magnet 45. In the second adjustment, the direction of the combined deflection force Ft is adjusted by adjusting the current to the deflection magnets 45A and 45B. In the second adjustment, fine adjustment is performed. In the structure shown in FIGS. 6A and 6B, the controller 7 adjusts the deflection direction by adjusting the rotation angle of the deflection magnet 45C. It is noted that these adjustments may be performed manually by an operator without the control of the controller 7.

It is noted that the configuration of the deflection magnet apparatus 30 described above may be applied to at least one of the deflection magnet apparatuses 30A, 30B, and 30C. It is noted that in FIG. 1, the deflection magnet apparatus 30A is described as having a pair of deflection magnets 40, but it may be configured as one deflection magnet 40 by adopting the structure shown in FIGS. 6A and 6B. Further, while the deflection magnet apparatuses 30B and 30C are described as having one deflection magnet, they may be configured as a pair of deflection magnets by adopting the structure shown in FIGS. 4A, 4B, and 4C and FIGS. 5A, 5B, and 5C.

The following describes the operation and effects of the particle beam treatment apparatus 1, the deflection magnet apparatus 30, and the particle beam adjustment method according to this embodiment.

According to the particle beam treatment apparatus 1, the deflection magnet 45 that deflects the particle beam B of the transport portion 20 is rotatable around the beam axis CL of the particle beam B. In this case, the deflection magnet 45 may be rotated around the beam axis CL to align with the desired deflection direction of the particle beam B. Thus, the direction in which the deflection force of the deflection magnet 45 is effectively obtained may be aligned with the desired deflection direction of the particle beam B. As a result, effective deflection force may be obtained regardless of the deflection direction of the particle beam B. It is noted that by obtaining an effective deflection force, it is possible to achieve the desired performance even with a smaller deflection magnet 45 compared to a non-rotating deflection magnet apparatus of a comparative example. In this way, cost down may be achieved by making the deflection magnet 45 smaller. Further, in the case of adjusting the rotation angle of the deflection magnet 45 itself, it is possible to reduce the number of adjustment process steps compared to adjusting the deflection direction solely by electrical methods, thereby enabling cost down.

The deflection magnet apparatus 30 includes a pair of deflection magnets 45A and 45B, and the pair of deflection magnets 45A and 45B may be rotatable around the beam axis CL of the particle beam B while being fixed to each other in the rotation direction. When combining the deflection forces of the pair of deflection magnets 45A and 45B, there are angles at which the combined deflection force may be secured at a large magnitude and angles at which the deflection force becomes small. For example, as shown in FIG. 3C, when the angle θ1 is 45°, the combined deflection force Ft may be maximized to the maximum deflection force Fmax, but when the angle θ1 is 0° or 90° the combined deflection force Ft may only be obtained up to the magnitude of one deflection force. In response to this, by rotating the pair of deflection magnets 45A and 45B around the beam axis CL of the particle beam B, it is possible to align the direction in which a large deflection force may be secured (for example, the direction in which the maximum deflection force Fmax is obtained) with the desired deflection direction of the particle beam B. Thus, regardless of the deflection direction of the particle beam B around the beam axis CL, a large deflection force of the pair of deflection magnets 45A and 45B may be secured. In this case, since the pair of deflection magnets 45A and 45B are fixed to each other in the rotation direction, the adjustment of the rotation angle may be performed more easily compared to individually adjusting the rotation angles of the pair of deflection magnets 45A and 45B.

The deflection magnet apparatus 30 includes a pair of deflection magnets 45A and 45B, and one of the pair of deflection magnets 45A and 45B may be rotatable around the beam axis of the particle beam independently from the other. When combining the deflection forces of the pair of deflection magnets 45A and 45B, there are angles at which the combined deflection force may be secured at a large magnitude and angles at which the deflection force becomes small. By rotating the pair of deflection magnets 45A and 45B around the beam axis of the particle beam, it is possible to align the direction in which a large deflection force may be secured with the desired deflection direction of the particle beam. Thus, regardless of the deflection direction of the particle beam B around the beam axis CL, a large deflection force of the pair of deflection magnets 45A and 45B may be secured. In this case, when combining the deflection force of one deflection magnet 45A with the deflection force of the other deflection magnet 45B, there exists a directional component where the two deflection forces cancel each other out (refer to the component Floss in FIG. 3C). In response to this, one of the pair of deflection magnets 45A and 45B is rotatable around the beam axis CL of the particle beam B independently from the other. As a result, the relative rotation angle of the deflection magnets 45A and 45B may be adjusted to minimize the directional components that cancel each other out. This allows for effective utilization of energy applied to the deflection magnet apparatus 30.

The deflection magnet apparatus 30 includes one deflection magnet 45C, and the one deflection magnet 45C may be rotatable around the beam axis CL of the particle beam B. In this case, the number of magnets in the deflection magnet apparatus 30 may be reduced.

The particle beam treatment apparatus 1 may include an adjusting mechanism 50 for adjusting the rotation angle of the deflection magnet 45 with respect to the beam axis CL. In this case, by using the adjusting mechanism 50, the rotation angle of the deflection magnet 45 may be adjusted easily and accurately.

The adjusting mechanism 50 may include a driver 60 for rotating the deflection magnet 45 with respect to the beam axis CL. In this case, the rotation angle of the deflection magnet 45 may be adjusted without manual operation by an operator.

The beam axis CL extends in the horizontal direction, and the orientation of the magnetic field of the deflection magnet 45 may be inclined with respect to the vertical direction.

The particle beam treatment apparatus 1 may further include a controller 7 for controlling the deflection magnet apparatus 30, and the controller 7 may perform a first adjustment of the particle beam B by rotating the deflection magnet 45. In this case, the controller 7 may roughly adjust the direction of the deflection force by rotating the deflection magnet 45 itself.

The controller 7 may perform a second adjustment of the particle beam B by electrically controlling the deflection magnet 45. In this case, the controller 7 may fine-tune the direction of the deflection force.

The deflection magnet apparatus 30 includes a pair of deflection magnets 45A and 45B, and in the first adjustment, the controller 7 may rotate the deflection magnets 45A and 45B relative to each other. In this case, the controller 7 may also adjust the angle between the pair of deflection magnets 45A and 45B in the first adjustment.

The deflection magnet apparatus 30 according to this embodiment is a deflection magnet apparatus 30 including a deflection magnet 45 for deflecting the particle beam B, and the deflection magnet 45 may be rotatable around the beam axis CL of the particle beam B.

The deflection magnet apparatus 30 may deflect the particle beam B in a desired direction by being provided where the particle beam B passes. At this time, the deflection magnet 45 may be rotated around the beam axis CL to align with the desired deflection direction of the particle beam B. Thus, the direction in which the deflection force of the deflection magnet 45 is effectively obtained may be aligned with the desired deflection direction of the particle beam B. As a result, effective deflection force may be obtained regardless of the deflection direction of the particle beam B.

The particle beam adjustment method according to this embodiment is a particle beam adjustment method for adjusting a deflection magnet that deflects the particle beam B, and the particle beam is adjusted by rotating the deflection magnet around the beam axis of the particle beam.

According to the particle beam adjustment method, the particle beam B may be deflected in a desired direction by providing the deflection magnet 45 where the particle beam B passes. At this time, the deflection magnet 45 is rotated around the beam axis CL to align with the desired deflection direction of the particle beam B. Thus, the direction in which the deflection force of the deflection magnet is effectively obtained may be aligned with the desired deflection direction of the particle beam B. As a result, effective deflection force may be obtained regardless of the deflection direction of the particle beam B.

The disclosure is not limited to the aforementioned embodiments.

In FIG. 1, although a cyclotron was exemplified as the accelerator, the configuration of the disclosure may be adopted for various accelerators such as synchrocyclotrons, linear accelerators (linacs).

In the above-described embodiment, although the deflection magnet apparatus 30 is applied to the transport portion of the particle beam treatment apparatus, the application is not particularly limited. For example, the deflection magnet apparatus 30 may be applied to high-energy accelerators. Further, the deflection magnet apparatus 30 may be applied to experimental facilities for charged particle beams. Further, the deflection magnet apparatus 30 may be applied to magnetic separators.

What is claimed is:

1. A particle beam treatment apparatus, comprising:
an irradiation portion configured to irradiate a particle beam to an irradiation target;
a transport portion configured to transport the particle beam; and
a deflection magnet apparatus having a deflection magnet for deflecting the particle beam in the transport portion,
wherein the deflection magnet is rotatable around a beam axis of the particle beam.

2. The particle beam treatment apparatus according to claim 1, wherein the deflection magnet apparatus has a pair of deflection magnets, and
the pair of deflection magnets are rotatable around a beam axis of the particle beam in a state where the deflection magnets are fixed to each other in a rotation direction.

3. The particle beam treatment apparatus according to claim 1, wherein the deflection magnet apparatus has a pair of deflection magnets, and
one of the pair of the deflection magnets is rotatable around a beam axis of the particle beam independently from other deflection magnet.

4. The particle beam treatment apparatus according to claim 1, wherein the deflection magnet apparatus comprises one deflection magnet, and
the one deflection magnet is rotatable around a beam axis of the particle beam.

5. The particle beam treatment apparatus according to claim 1, further comprising an adjusting mechanism configured to adjust a rotation angle of the deflection magnet with respect to the beam axis.

6. The particle beam treatment apparatus according to claim 5, wherein the adjusting mechanism comprises a driver for rotating the deflection magnet with respect to the beam axis.

7. The particle beam treatment apparatus according to claim 2, wherein the beam axis extends in a horizontal direction, and an orientation of magnetic field of the deflection magnet is inclined with respect to a vertical direction.

8. The particle beam treatment apparatus according to claim 4, wherein the beam axis extends in a horizontal direction, and an orientation of magnetic field of the deflection magnet is inclined with respect to a vertical direction.

9. The particle beam treatment apparatus according to claim 1, further comprising: a controller configured to control the deflection magnet apparatus,
wherein the controller performs a first adjustment of the particle beam by rotating the deflection magnet.

10. The particle beam treatment apparatus according to claim 9, wherein the controller performs a second adjustment of the particle beam by electrically controlling the deflection magnet.

11. The particle beam treatment apparatus according to claim 9, wherein the deflection magnet apparatus has a pair of deflection magnets, and
in the first adjustment, the controller rotates the deflection magnets relative to each other.

12. A deflection magnet apparatus comprising a deflection magnet that deflects a particle beam, wherein
the deflection magnet is rotatable around a beam axis of the particle beam.

13. A particle beam adjustment method for adjusting a deflection magnet that deflects a particle beam, the particle beam adjustment method comprising:
adjusting the particle beam by rotating the deflection magnet around a beam axis of the particle beam.
